(19) United States
(12) Patent Application Publication
(10) Pub. No.: US 2024/0417661 A1
(43) Pub. Date: Dec. 19, 2024

(54) MICROFLUIDIC CELL CULTURING DEVICE

(71) Applicant: UNIVERSITÄT BERN, Bern (CH)

(72) Inventors: Olivier Thierry GUENAT, Bollingen (CH); Soheila ZEINALI, Bern (CH); Rrahim GASHI, Zofingen (CH); Karin Farah RECHBERGER, Solothurn (CH)

(21) Appl. No.: 18/705,292

(22) PCT Filed: Oct. 28, 2022
(86) PCT No.: PCT/EP2022/080214

§ 371 Date: Apr. 26, 2024

(30) Foreign Application Priority Data
Oct. 29, 2021 (CH) 070471/2021

(51) Int. Cl. C12M 3/06; C12M 1/00; C12M 3/00

(52) U.S. Cl. CPC C12M 23/16; C12M 21/08; C12M 23/22; C12M 29/10

(57) ABSTRACT

A microfluidic cell culturing device comprises a cell culture cavity, a first perfusion channel having an inlet and an outlet, a first capillary pressure barrier essentially vertically connecting the first perfusion channel with the cell culture cavity, a second perfusion channel having an inlet and an outlet, and a second capillary pressure barrier essentially vertically connecting the second perfusion channel with the cell culture cavity.

### TECHNICAL FIELD

The present invention relates to a microfluidic cell culturing device that enables, for example, the creation of a self-assembled vascular network and/or the creation of vascularized tissues, such as spheroids, organoids and tissue biopsies. It may also provide a method to create various cell culture compartments that can communicate with each other, for instance to mimic the interactions between different tissues.

### BACKGROUND ART

Blood vessel formation is a key process in organogenesis. The formation of blood vessels takes place through angiogenesis, intussusception, and/or vasculogenesis. While intussusception is the splitting of existing vessels in two, vasculogenesis is the self-assembly of the blood vessels from vascular cells and angiogenesis is the formation of the new vessels from pre-existing parental vessels. These mechanisms depend on the ability of vascular cells to migrate while remaining firmly attached to each other. Endothelial cells covering the interior surface of blood vessels, adopt organ-specific functions and characteristics that will guide the tissue development.

Blood and lymphatic vessels are fundamental elements of the human body. Blood vessels serve a wide range of functions from nutrients and oxygen delivery, cellular and biochemical substances transport, removal of waste cellular products and carbon dioxide. Blood vessels functions can importantly be impaired, by the loss of the integrity of the vessels or their inability to contract or expand in response to biochemical or physical stimuli.

The development and the remodeling of blood vessels are major aspects of many physiological and pathological processes. The endothelium that lines the blood vessel barrier finely controls the passive transport of fluids, solutes and macromolecules between the vessels and the surrounding tissues by interstitial and/or osmotic pressure gradients. It aims at providing tissues with nutrients and oxygen to preserve their homeostasis. When this equilibrium is disturbed due to a pathological condition, such as inflammation or infection, gaps form in the endothelial layer, leading to an increased barrier permeability, an accumulation of fluids in the extracellular space and ultimately to the formation of an edema.

The lymphatic network originates near the capillary beds as blind-ended initial lymphatics, which are characterized by a thin single layer of lymphatic endothelial cells and a discontinuous basal membrane. This highly permeable initial lymphatics facilitates the drainage of interstitial fluid and macromolecules from the extracellular space and thus provides maintenance of the tissue fluid balance. Initial lymphatics are interconnected with collecting lymphatic vessels that drain the lymph into regional lymph nodes for immunologic surveillance of the lymph prior return to the venous system.

Increased interstitial fluid pressure due to disrupted vascular endothelial barrier in cancer can facilitate the invasion of cancer cells into the lymphatic system, where malignant cells reach sentinel lymph nodes before disseminating to distant organs via blood circulation.

In this connection, several microfluidic-based microvasculature systems have been reported in the past few years. They enable the creation of three-dimensional networks of functional blood vessels. Often these systems are used to test drugs on the vessel formed, investigate intravasation and extravasation of cells, such as cancer circulating cells, or mimic complex biological processes, such as infection. These systems can also be used to create vascularized tissues. In such cases, the vasculature network created is used as vascular bed on which a tissue (spheroid, organoid) is placed and vascularized.

These systems are either based on patterned hydrogel tubes in which endothelial cells are seeded, or on the self-assembly of vascular cells, mostly a mixture of endothelial and mural cells within an extracellular matrix made of hydrogel. In the latter case, the hydrogel is typically mixed with vascular cells, and introduced in the microfluidic device by capillary forces and/or the pipetting force. The mixture of the hydrogel and the cells is maintained in a geometrically defined compartment. The mixture is confined in these compartments by means of capillary valves or capillary pressure barriers. In short, hydrogel flow is stopped when its liquid-air meniscus is pinned at a certain position by capillary forces. Once the hydrogel flow stops, the hydrogel is allowed to gelate and thus is maintained in this position. Usually, an array of capillary valves is used to delimit the microfluidic compartment. The capillary valves are located at the interface between the hydrogel compartment and one or more adjacent channels that are filled with physiological medium and oxygen to nurture and oxygenize the cells in the hydrogel matrix.

So far two main types of capillary valves or capillary pressure barriers have been reported. They are based on specific microstructures located at the interface between the hydrogel compartment and the adjacent channel: a) micropillar and array and b) phase-guide. Yet another type relates to wedge-guide structures.

In general, the mechanism of capillary valves is well known. It typically is either based on an abrupt geometrical change of the channel in which the hydrogel flows or on modification of the surface tension properties in the fluidic path in which the liquid propagates. When the liquid reaches this location, the contact angle of the liquid and the fluidic path increases, which stops the flow following a decrease of the radius of the moving meniscus and thus an increase in surface tension pressure, as described by Young Laplace law.

More specifically, devices are known where one or more channels can be filled with a biologically relevant gel, such as collagen, which is held in place by an array of posts. The posts are tiny microstructured pillars, typically separated by 120 mm, located at the interface between the hydrogel and an adjacent channel. The devices can then be plated with endothelial cells and new blood vessels grow in the gel.

Similar devices are described in the art having several fluidic compartments in which hydrogel can be maintained by capillary valves based on arrays of micropillars. In addition, an array of holes that separates the vessel compartment and another compartment that can be filled with a tissue, such as a spheroid can be provided.

In another known device, a microfluidic network is used that comprises a capillary pressure barrier to stop the progression of the hydrogel flow. The capillary pressure barrier extends longitudinally along two adjacent microfluidic channels. This capillary pressure barrier can be located at different heights, as described in WO 2021/216848 A1 (phase guide system).

Another system described in WO 2012/050981 A1 relies on an array of trapezoidal posts that confine the gel by creating capillary barriers between posts. These trapezoidal posts limit the interface area between the gel and the physiological medium, resulting in a reduced space for the vessels to form (only between the posts).

In a further example, WO 2017/216113 A2 (phase guide system) describes a multi-well plate allowing for a controlled and reliable vascularization and/or perfusion of organoids assays. The device comprises one or several capillary pressure barriers allowing for the formation of an extracellular matrix gel within a confined area of the network, in which cells can be cultured for different uses.

In yet another example, US 2020/0156062 A1 (wedge guide system) describes a microfluidic device in which microfluidic channels are embedded in a cell culture well and have open sides. The microfluidic device is patterned with a fluid moved along a hydrophilic surface due to capillary force, and the fluid may be rapidly and uniformly patterned along an inner corner path and a microfluidic channel. In the microfluidic device, the microfluidic channel is connected to facilitate fluid flow with a culture medium through open sides thereof and openings, and thus may provide a cell culture environment in which high gas saturation is maintained.

Although much has been reported about the functional vascular network vascularisation of organoids and spheroids, much remains to be made to mimic the true vascularisation and create complex vascularized tissues. Therefore, there is a need for a device allowing to mimic true vascularisation and/or to create complex vascularized tissues.

### DISCLOSURE OF THE INVENTION

According to the invention this need is settled by a microfluidic cell culturing device as it is defined by the features of independent claim 1, and by use of such a microfluidic cell culturing device as it is defined by the features of independent claim 33. Preferred embodiments are subject of the dependent claims.

In one aspect, the invention is a microfluidic cell culturing device comprising a cell culture cavity, a first perfusion channel having an inlet and an outlet, a first capillary pressure barrier essentially vertically connecting the first perfusion channel with the cell culture cavity, a second perfusion channel having an inlet and an outlet, and a second capillary pressure barrier essentially vertically connecting the second perfusion channel with the cell culture cavity.

The term “microfluidic” as used in connection with the invention relates to structures and volumes or flows in sub-milli or small milli dimensions or lower. For example, a compartment of the microfluidic device can be in a range of 10 millimeters (mm) or lower such as about 2 mm to about 5 mm in diameter, and, e.g., about 50 micrometers (μm) to about 500 μm in height. An advantageously dimensioned microvasculature compartment, e.g. embodied in the cell culture cavity, can have a diameter of about 3 mm and a height of about 200 μm. Such microvasculature compartment can, however, have various shapes, it can for instance be rectangular with a width of about 0.5 mm to about 3 mm and a length of about 1 mm to about 5 mm. Of course, other and particularly similar dimensions may also be appropriate.

The term “compartment” as used herein may relate to a more or less closed cavity. It may still have openings and/or accesses to channels but for the major part it is closed. Further, a compartment typically is dimensioned to have significant extensions in all horizontal directions. In contrast thereto, a channel typically has a comparably large extension on one usually horizontal direction stablishing a length of the channel, and a comparably small extension in another horizontal direction establishing a width of the channel. Also, often channels are intended to house a liquid or gas flowing at a comparably high speed whereas compartments house a liquid or gas not flowing or flowing at a comparably low speed.

The first and second first perfusion channels may be embodied as microfluidic perfusion channels. Generally, a “microfluidic channel” may be a channel on or through a layer of material that can be covered by a top-substrate or cover, with at least one of the dimensions of length, width or height being in the sub-mm or small mm range. It will be understood that the term encompasses channels which are linear channels, as well as channels which are branched, or have bends or corners within their path. A microfluidic channel typically and the first and second perfusion channels specifically comprise an inlet for administering a volume of liquid.

A volume enclosed by a microfluidic channel or a compartment is typically in the microliter or sub-microliter range. A microfluidic channel typically comprises a base, which may be the top surface of an underlying material, two side walls, and a top wall, which may be the lower surface of a top substrate overlying the microfluidic channel, with any configuration of inlets, outlets and/or vents as required.

The term “vertical” as used in connection with the first and second capillary pressure barriers relates to an orientation of the microfluidic cell culturing device when being applied or used as intended. It is understood that also in application or use as intended, the microfluidic cell culturing device can be oriented such that vertically oriented elements or structures are temporarily not in a vertical direction in the literal sense.

For being stably positionable as intended, the microfluidic cell culturing device preferably has a bottom side essentially horizontally extending. More specifically, the bottom side preferably is configured such that, in use, the microfluidic cell culturing device seats on the bottom side. By such configuration the microfluidic cell culturing device can efficiently be positioned and oriented as intended.

Herein, the terms “capillary valve” and “capillary pressure barrier” are used interchangeably. They typically are used in reference to features that keep a liquid-air or liquid-liquid meniscus pinned on a certain position by capillary forces. In general, these terms are relating to barriers involving capillary action, i.e. a process of a liquid flowing in narrow spaces without the assistance of, or even in opposition to, external forces like gravity. Such action typically occurs because of intermolecular forces between the liquid and surrounding solid surfaces. If the diameter of the surrounding solid surfaces is sufficiently small, then the combination of surface tension (which is caused by cohesion within the liquid) and adhesive forces between the liquid and the surrounding solid surfaces act to propel the liquid.

By having at least the first and second perfusion channels connected to the cell culturing cavity, nutrients and oxygen can be provided by means of a cell culture medium to cells cultured in the cell culturing cavity and remove waste products. As used herein, the term “cell culture medium” or, in some instances, brief “medium” relates or corresponds to “physiological medium”.

In a minimum setting, one of the first and second channels with its inlet is connected to one horizontal side of the cell culturing cavity and its prolongation to the other horizontal side of the cell culturing cavity where it is connected to its outlet. On each side of the cell culturing cavity, the perfusion channels may be connected to an air vent, with dimensions small enough to selectively allow air bubbles to get extracted from the respective perfusion channel while blocking the flow of the cell culture medium. One or more inlets or outlets can be added to the perfusion channels for instance to create a gradient of growth factors such as vascular endothelial growth factor (VEGF) across the cell culturing cavity to favour the growth the vessels and their maturation.

One or more perfusion channels are needed to provide nutrients and oxygen to the cells cultured in the microvasculature compartment and remove their waste products. In the minimum setting, a channel with an inlet is connected to one side of the microvasculature compartment and its prolongation to the other side of the microvasculature compartment where it is connected to an outlet. On each side of the microvasculature compartment, the perfusion channel may be connected to an air vent, with dimensions small enough to selectively allow air bubbles to get extracted from the perfusion channel while blocking the flow of the cell culture medium. One or more inlets or outlets can be added to the perfusion channels for instance to create a gradient of growth factors such as VEGF across the microvasculature compartment to favor the growth the vessels and their maturation.

In use, during the first few days after cell seeding, the cells start to self-assemble in 3D vessels, and only an interstitial pressure can be applied across the cell culturing cavity between the two perfusion channels by creating a hydrostatic pressure difference. Once the vasculature is perfusable, a flow of physiological medium can be produced across the vasculature network, which brings additional nutrients and oxygen to the cells. In case the vasculature does not form into perfusable vessels, which can be the case with diseased endothelial cells, then the nutrients are only brought to the cells by the perfusion channels through interstitial flow.

The microfluidic cell culturing device can particularly be beneficially used for vascularisation of tissues. Such tissue vascularisation relates to reproducing in-vivo conditions of part of the vasculature of a tissue, such as, e.g., the lung, the liver, the brain, and more particularly to tissue interfaces such as tissue interfaces between an epithelial barrier and the vascular or the lymphatic endothelium.

As used herein, the term “tissue” refers to a collection of identical, similar or different types of functionally interconnected cells. Particularly, it can relate to such cells that are to be cultured and/or assayed in methods or processes described herein. The cells may be a single cell, a cell aggregate, or a particular tissue sample from a patient. For example, the tissue encompasses spheroids, organoids, a cellular barrier (e.g. epithelium) such as endothelial and/or epithelial cells, tissue biopsies, tumor tissue, resected tissue material and embryonic bodies. The type of cells can either be from a specific organ, such as lung microvascular endothelial cell, or can be a stem cell or an induced pluripotent stem cells that can differentiate in a specific phenotype.

A cell aggregate can be a spheroid, an organoid, a tissue explant such as a biopsy or a precision-cut slice of a specific organ. The vascularized tissue can be exposed to specific agents such as drug, pathogens, chemicals or the like to change or maintain its functions that in turn can be extracted using biotechnological techniques such as PCR, single cell RNA sequencing, imaging by fluorescent or conventional microscopy, etc.

As used herein, the term “endothelial cells” refers to cells of endothelial origin or cells that are differentiated into a state in which they express markers identifying the cell as an endothelial cell. This includes induced pluripotent stem cells derived endothelial cells or embryonic derived endothelial cells.

Similarly, the term “epithelial cells” refers to cells of epithelial origin, or cells that are differentiated into a state in which they express markers identifying the cell as an epithelial cell. This includes induced pluripotent stem cells derived epithelial cells or embryonic derived epithelial cells.

As used herein, the term “cell aggregate” refers to a three-dimensional (3D) cluster of cells in contrast with surface attached cells that typically grow in monolayers. 3D clusters of cells are typically associated with a more in-vivo like situation. In contrast, surface attached cells may be strongly influenced by the properties of the substrate and may undergo de-differentiation or undergo transition to other cell types.

As used herein, the term “organoid” refers to a miniature form of a tissue that is generated in vitro and exhibits endogenous 3D organ architecture.

The term “cell culture cavity” as used in connection with invention relates to a hollow space in the interior of the microfluidic cell culturing device. In particular, such cavity may be specifically be structured and configured to allow efficient growing of cells as well as treatment of cells. The hollow space may be open or widely closed. Thereby, an open hollow space may still be covered, e.g. by a lid. It can further be configured to receive a medium such as a hydrogel comprising cells or not. The hydrogel can be a gel, such as fibrin gel, collagen, elastin, Matrigel or the like, typically containing cells, such as endothelial cells and/or mural cells.

Typically, endothelial cells (such as HUVEC, tissue-specific endothelial cells or induced pluripotent stem cells-derived endothelial cells) and mural cells (such as tissue-specific fibroblasts or pericytes, smooth muscle cells, mesenchymal cells) are mixed at a specific ratio (typically 1:2) they self-assemble into structures of higher-order and create lumen that is perfusable after about 5 days. In addition to mixing two different cell types (endothelial and mural cells) for vessel self-assembly, three or more different cell types can be mixed with hydrogel at a specific ratio and seeded in the microvasculature compartment. For instance, mixture of the endothelial, mural cells with smooth muscle cells or cardiomyocytes or hepatocytes, or tumor cells can be considered. The cell types, the cell density, and the cell culture ratio, and their health status (healthy, diseased, etc.) all affect importantly the vessels size, morphology, permeability, vasoactive response, the vascularized area and the number of nodes within the network and the vascular network interconnectivity and perfusability.

The cell culture cavity can be embodied to be at least partially filled with a hydrogel prior to its jellification. Either a hydrogel channel with an access hole in the second body portion or an additional cell culture chamber in the second body portion providing the microvasculature compartment is fluidically connected to this cell culture cavity. An additional hydrogel channel with its access hole is designed to allow excess hydrogel to exit the microvasculature compartment

By connecting the first and second perfusion channels to the cell culture cavity via the first and second capillary pressure barriers, the interior of the cell culture cavity and particularly cells in the cell culture cavity can appropriately be perfused by a fluid circulating through the first and second perfusion channels. The fluid may contain air, blood, cell culture medium, hydrogel, cells, water, compounds, particulates and/or any other media to be delivered to the cell culture cavity.

Compared to the systems known in the art, the microfluidic cell culturing device is based on a capillary valve or capillary pressure barrier created in the z-direction or close to the z-direction. In addition, unlike known systems, based on the use of the capillary pressure barriers, no microstructured pillars, phase guides, collars or the like are necessary. In an efficient manner, the barriers can be created by two adjacent cavities created opposite each other in the z-direction and slightly misaligned in the x-y-direction such that produce an appropriately dimensioned aperture is created. Thus, the dimensions of the aperture created are defined by the geometry of the cavities and the x-y-misalignment of the two cavities. For example, a typical size of such apertures may be in a range from about 50 μm to about 500 μm, so that they can act as a capillary pressure barrier that can be calculated by the Young Laplace law.

In general, cells such as endothelia cells, fibroblasts, and the like proliferate faster and form a monolayer on substrates which are stiffer than hydrogel. For instance, in known systems of the art, the interface between the vasculature and graft compartments is a phase guide feature which is made of comparably hard plastic. Within the processes of endothelial barrier formation and vascular sprouting, endothelial cells or the other cell types, are supposed to proliferate on its surface instead of forming 3D vasculature. However, in accordance with the invention, the interface of the microvasculature network and organoid/spheroid can be a uniform layer of hydrogel. Hard plastic or glass interface features are not necessary.

Compared to the systems known in the art, the microfluidic cell culturing device is suitable to be implemented with many different types of hydrogels. For example, hydrogels without cross-linker which hydrogel viscosity remains the same within time (different concentrations of the different collagen types), and others with cross-linker which the hydrogel viscosity increases with time (mixture of the fibrinogen solution with thrombin which results in formation of the fibrin) can be used in the same device. Once fibrinogen is mixed with thrombin, fibrin formation happens very fast (depending on the concentration the time varies from few seconds to a minute). Recently, it has been proven that fibrin is angiogenesis compound and provides several advantages once used in angiogenesis and vasculogenesis assays. Therefore, the microfluidic cell culturing device having flexibility for such implementation is beneficial. To the contrary, in known system, typically only hydrogels with constant viscosity can be used. For example, in these systems seeding fibrin usually will not result in formation of a uniform hydrogel wall because the gel may stuck somewhere or pass across the phase guide in the case of higher seeding pressure.

Vasculogenesis and angiogenesis assays which can be implemented in the microfluidic cell culturing device are often physiologically more relevant. Monoculture of endothelial cells or co-culture of endothelial cells and mural cells may be mixed with hydrogel and seeded in the microvasculature compartment. To induce microvasculature self-assembly, angiogenesis boosters as required in known systems may not be necessary and vasculogenesis/angiogenesis can efficiently take places from cells by standard cell culture media like EGM2 or EMG2-MV. Using such angiogenesis cocktail may be undesired as it can affect the function of the microvasculature network.

Also, the microfluidic cell culturing device may not need any external pump or hardware (tilter) as microvasculature self-assembly from cells is enabled. In known system, it may be required first to obtain confluent endothelial walls and second to induce sprouting from that which requires the system to be placed on an external rocker applying back and forth (non-physiological) flow (shear stress) on endothelial cells. In addition to complexity regarding vasculogenesis/angiogenesis assays, the cells may undergo non-physiological flow which is shown induces pro-inflammatory response on endothelial cells.

Further, in order to increase the interface area connecting the medium, e.g. a hydrogel, of the cell culturing cavity and the medium, e.g. a physiological medium aiming at nurturing the cells contained in the cell culturing cavity, advantageously plural apertures between the cell culturing cavity and the respective perfusion channel is provided.

Further, as mentioned, the aperture establishing the capillary pressure barrier can be provided at the interface of two plates facing each other, wherein the cell culturing cavity and the respective perfusion channel are slightly horizontally misaligned, i.e. in the x-y direction.

In summary, the microfluidic cell culturing device according to the invention allows for a comparably simple and efficient construction particularly as to the capillary pressure barrier. Moreover, it allows for an accurate and efficient application particularly for vascularisation of tissues.

In contrast to the known phase-guide systems and wedge-guide system, the present innovation can also be used regardless of the surface properties of the microfluidic chip (i.e. the chip can be successfully loaded regardless of whether the surface is hydrophilic or hydrophobic). If the surface is hydrophilic, the microfluidic chip can be loaded with gel by capillary forces, if it is hydrophobic the gel can be loaded on the chip with pressure induced by the pipette. Another benefit is that the present invention enables the loading of the chip with gel of varying viscosity, such as with a mixture of fibrinogen solution and thrombin. In the latter, the viscosity changes rapidly once the two compounds are mixed which results in a fibrin gel.

The microfluidic cell culture device can be used for preclinical use such as drug discovery and investigation of pathophysiological effects, where it can be used as cell assay device to screen for agents that influence living cells. For instance, the microfluidic cell culture device can be used to screen for anti-angiogenesis agents, anti-metastasis agents, wound healing agents and tissue engineering agents.

The microfluidic cell culture device can also be implemented for clinical use, such as precision medicine or regenerative medicine. For instance, the microfluidic cell culture device can be used as an in-vitro test for new anti-cancer drugs and to match the best anti-cancer drug to a particular patient in a hospital setting. It can also be used in view to culture cells and/or reconstruct tissues aimed for transplantation purposes.

The microfluidic cell culture device can be embodied to be tilted in the z-direction along a lateral axis to create a hydrostatic pressure difference between the inlet of the first perfusion channel and the outlet of the second perfusion channel. The outlet of the first perfusion channel and the inlet of the second perfusion channel may need to be closed to enable a flow across the cell culturing cavity, which is then exposed to a mechanical shear stress.

In a non-limiting example embodiment, the microfluidic cell culture device allows simulation of the structure and function of a functional alveolar-capillary unit that is vascularized with a dense network of small capillaries. The microfluidic cell culture device can be used to simulate air-borne and blood-borne chemical, molecular, particulate and cellular stimuli to investigate the exchange of chemicals, molecules, and cells across this tissue-tissue interface through a vasculature to the alveolar epithelium. The microfluidic cell culture device may impact the development of in-vitro lung models that mimic organ-level responses, which are able to be analyzed under physiological and pathological conditions.

In another non-limiting example embodiment, the microfluidic cell culture device can be configured to mimic operation of lung adenocarcinoma, whereby lung tumor cells either intravasate from the main tumor site or extravasate across the endothelial barrier of the vasculature to create a metastatic niche. Antineoplastic drugs and even cancer immunotherapies can be simulated with the device with immune cells that can be added to the model.

Further embodiments of the microfluidic cell culture device can be applied in numerous fields including basic biological science, life science research, drug discovery and development, drug safety testing, toxicology, chemical and biological assays, as well as tissue and organ engineering. In an embodiment, the microfluidic cell culture device is a bioartificial organ device which can be used as organ-specific disease biology. Furthermore, the microfluidic cell culture device can find application in organ assist devices for liver, kidney, lung, intestine, bone marrow, and other organs and tissues, as well as in organ replacement structures.

Applications of the microfluidic cell culture device may also include, but are not limited to, identification of markers of disease; assessing efficacy of anti-cancer therapeutics; testing gene therapy vectors; drug development; screening; studies of cells, especially stem cells and bone marrow cells; studies on biotransformation, absorption, clearance, metabolism, and activation of xenobiotics as well as drug delivery; studies on bioavailability and transport of chemical or biological agents across epithelial layers as in the intestine and the lung, endothelial layers as in blood vessels, and across the blood-brain barrier can also be studied; studies on transport of biological or chemical agents across the blood-brain barrier; studies on transport of biological or chemical agents across the intestinal epithelial harrier; studies on acute basal toxicity of chemical agents; studies on acute local or acute organ-specific toxicity of chemical agents; studies on chronic basal toxicity of chemical agents; studies on chronic local or chronic organ-specific toxicity of chemical agents; inhalation toxicity studies; repeated dose toxicity studies; long-term toxicity studies; chronic toxicity studies; studies on teratogenicity of chemical agents; studies on genotoxicity, carcinogenicity, and mutagenicity of chemical agents; detection of infectious biological agents and biological weapons; detection of harmful chemical agents and chemical capons; studies on infectious diseases and the efficacy of chemical and biological agents to treat these diseases, as well as optimal dosage ranges for these agents can be studied; studies on the efficacy of chemical or biological agents to treat disease; studies on the optimal dose range of agents to treat disease; prediction of the response of organs in-vivo to chemical and biological agents; prediction of the pharmacokinetics of chemical or biological agents; prediction of the pharmacodynamics of chemical or biological agents studies concerning the impact of genetic content on response to agents; studies on gene transcription in response to chemical or biological agents; studies on protein expression in response to chemical or biological agents; and studies on changes in metabolism in response to chemical or biological agents.

In specific applications, the microfluidic cell culture device can particularly be advantageous. E.g., some of the advantages of the device, as opposed to conventional cell cultures or tissue cultures, can for instance include, when cells are placed in the device, fibroblast, smooth muscle cell (SMC) and endothelial cell (EC) differentiation can occur that re-establishes a defined three-dimensional architectural tissue-tissue relationships that are close to the in-vivo situation, and cell functions and responses to pharmacological agents or active substances or products can be investigated at the tissue and organ levels. In addition, many cellular or tissue activities are amenable to detection in the device, including, but not limited to diffusion rate of the drugs into and through the layered tissues in transported flow channel; cell morphology, differentiation and secretion changes at different layers; cell migration, locomotion, growth, apoptosis and the like. Further, the effect of various drugs on different types of cells located at different layers of the system may be assessed easily.

The microfluidic cell culture device can be employed in engineering a variety of tissues including, but not limited to the cardiovascular system, lung, intestine, kidney, brain, bone marrow, bones, teeth, and skin. If the device is fabricated with a suitable biocompatible and/or biodegradable material, such as poly-lactide-co-glycolide acid (PLGA), it may be used for transplantation or implantation in-vivo. Moreover, the ability to spatially localize and control interactions of several cell types presents an opportunity to engineer hierarchically, and to create more physiologically correct tissue and organ analogs. The arrangement of multiple cell types in defined arrangement has beneficial effects on cell differentiation, maintenance and functional longevity.

For drug discovery, for example, there can be two advantages for using the microfluidic cell culture device: (1) the microfluidic cell culture device may be better able to mimic in-vivo layered architecture of tissues and therefore allows one to study drug effect at the organ level in addition to at the cellular and tissue levels; and 2) the microfluidic cell culture device may decrease the use of in-vivo tissue models and the use of animals for drug selection and toxicology studies.

In addition to drug discovery and development, the device according to the invention may be also useful in basic and clinical research. For example, it can be used to research the mechanism of tumorigenesis. It is well established that in-vivo cancer progression is modulated by the host and the tumor micro-environment, including the stromal cells and extracellular matrix (ECM). For example, stromal cells were found being able to convert benign epithelial cells to malignant cells, thereby ECM was found to affect the tumor formation. There is growing evidence that cells growing in defined architecture are more resistant to cytotoxic agents than cells in mono layers. Therefore, the device may be a better means for simulating the original growth characteristics of cancer cells and thereby better reflects the real drug's sensitivity of in-vivo tumors.

In yet another aspect, the microfluidic cell culture device can be used in understanding fundamental processes in cell biology and cell-ECM interactions. The in-vivo remodelling process is a complicated, dynamic, reciprocal process between cells and ECMs. The device can be able to capture the complexity of these biological systems, rendering these systems amenable to investigation and beneficial manipulation. Furthermore, coupled with imaging tools, such as fluorescence microscopy, live cell imaging, microfluorimetry or optical coherence tomography (OCT), trans-epithelial electrical resistance (TEER), real-time analysis of cellular behavior in the multilayered tissues is expected using the device. Examples of cell and tissue studies amenable to real-time analysis include cell secretion and signalling, cell-cell interactions, tissue-tissue interactions, dynamic engineered tissue construction and monitoring, structure-function investigations in tissue engineering, and the process of cell remodelling matrices in-vitro.

In another configuration, the microfluidic cell culture device can be perfused to generate a flow of physiological medium across the vascularized tissue (cellular aggregates, organoid or spheroid), made of a vascular bed on top of which a cellular aggregate, an organoid, a spheroid or a cellular barrier is cultured. The generation of the medium flow can be passive or active. Passive by using a hydrostatic pressure between two channels of the cell culture media. Active by using one or more internal, e.g. directly integrated in the microfluidic cell culture device, or external pumps to the perfusion channels. This may allow investigating the effect of the shear stress induced by the flow on the blood vessels and on the viability, size and functions of the vascularized tissue.

Preferably, the cell culture cavity has a compartment portion. Such compartment portion allows for efficiently providing room for growing and culturing the cells. For example, such compartment portion may house a hydrogel comprising cells or a vasculature network to be cultured.

For an efficient delivery of media to the compartment portion, preferably the first capillary pressure barrier essentially vertically connects the first perfusion channel with the compartment portion of the cell culture cavity. Additionally or alternatively, preferably the second capillary pressure barrier essentially vertically connects the second perfusion channel with the compartment portion of the cell culture cavity. By means of this vertical connection, large interface areas respectively apertures are provided which enable the formation of vessels across these interfaces, leading to an increased probability of network perfusability. These apertures have a larger surface area than those provided by phase-guide, wedge-guide or similar systems as the height of these systems are often limited to typical height of microfluidic channels (often between 50 micrometers to 200 micrometers, typically 100 micrometers). In the present invention, the aperture being in the XY plane is only limited by Young-Laplace law and can thus be larger (typically, 500 (width)×1300 (length) micrometers).

Preferably, the cell culturing cavity comprises a neighboring compartment portion adjacent to the compartment portion. Thereby, the microfluidic culturing device preferably comprises a capillary pressure compartment barrier essentially vertically connecting the compartment portion and the neighboring compartment portion. The capillary pressure compartment barrier may comprise a capillary member with a hole as described below in connection with the first and second capillary pressure barriers. In general, the capillary member may be any body or body portion having the hole dimensioned to implement the capillary effect. For example, the capillary member can be a sheet such as a plate or a foil.

The cell culturing cavity with the compartment portion and neighboring compartment portion may enable perfusion of the tissue, such as a vascularized epithelial barrier. As an example, epithelial cells aimed at forming an epithelial barrier may be introduced on top of a vascular bed by providing them through neighboring compartment portion. Once the cells are introduced, a flow can be stopped to allow the cells to adhere on the hydrogel in the compartment portion or the microvasculature therein. After the cells adhere on the hydrogel, they can be perfused and may be exposed to shear stress of the flow. It may be envisaged that epithelial barriers, such as of the lung (airways and alveolar), the gut, the skin, the kidney, the bladder, etc. can be reproduced like this. Also, instead of cell culture medium, an air-liquid interface can be created that would be of interest, e.g., for the lung.

The microfluidic cell culturing device can be embodied as a single piece unit. For example, it can be additive manufactured or injection molded. However, for an increased flexibility and often simpler set-up, the microfluidic cell culturing device can be a multi-component construction, e.g., formed by plural body portions. Thereby, the body portions can be formed as distinct physical units which are mounted together and, eventually, can be de-mounted if required. The body portions can, e.g., be essentially plate-shaped.

Preferably, the microfluidic cell culturing device comprises a culture plate and a perfusion plate, wherein the culture plate is equipped with the compartment portion of the cell culture cavity and the perfusion plate is equipped with the first perfusion channel and the second perfusion channel.

The term “plate” can relate to a plate in the literal sense, i.e., a flat piece of a substrate or material that is comparably hard and does not essentially bend. It can also relate to body or body portion being thicker or bulkier than a typical plate. Advantageously, the plate is a microplate made of a biocompatible material such as polystyrene (PS), polycarbonate (PC), poly methyl methacrylate (PMMA), cyclic olefin copolymer (COC), cyclic olefin polymers (COP) and the like.

By having the culture and perfusion plates, the microfluidic cell culturing device can efficiently and accurately be manufactured. In particular, the culture plate can specifically be structured to embody the compartment portion and, likewise, the perfusion plate the first and second perfusion channels.

Thereby, the culture plate preferably is mounted on top of the perfusion plate or the perfusion plate is mounted on top of the culture plate.

The culture plate preferably abuts the perfusion plate. In other words, the two plates are stacked on each other which allows for an efficient assembly and connecting.

Preferably, the microfluidic cell culturing device further comprises a bottom plate which advantageously is transparent for microscopic inspection. Such bottom plate allows to provide a solid stand for the device. Further, it can establish a bottom of the cell culturing cavity and/or the perfusion channels.

Thereby, the bottom plate abuts the cell culture plate such that it can be stacked with the cell culture plate.

Preferably, the first capillary pressure barrier has a first vertical passage between the first perfusion channel and the cell culture cavity, and the second capillary pressure barrier has a second vertical passage between the second perfusion channel and the cell culture cavity. The vertical passage can particularly be shaped and dimensioned for allowing generation of capillary action such that the capillary pressure barrier is established. When including the culture and perfusion plate, the apertures can be formed by the perfusion channels slightly overlying the cell culturing cavity.

Preferably, the microfluidic cell culturing device comprises an inflow medium channel which has an access hole and which opens into the cell culture cavity. It can particularly open into the compartment portion of the cell culture cavity. Like this, the cell culture cavity can at least partially be filled with a hydrogel prior to its jellification or another medium through the inflow medium channel. More specifically, the hydrogel can be introduced with a pipette in the access hole of the inflow medium channel. The gel may spread in the channel and, at least partially, fill the cell culturing cavity by the action of surface tension force and of the pressure created by the pipette. The gel flow stops when it reaches the aperture at the interface of the two cavities defined earlier creating the capillary pressure barrier. It may then gelate and the cells within the gel can start to migrate and self-assemble.

Alternatively, the hydrogel can be provided via an additional cell culture chamber fluidically connected to the cell culture cavity or via a direct contact between adjacent chambers.

Preferably, the microfluidic cell culturing device comprises an outflow medium channel which has an access hole and which opens into the cell culture cavity. Such outflow medium channel allows for exiting the hydrogel or other medium from the cell culturing cavity.

Preferably, the cell culture cavity has a transparent bottom. For example, the transparent bottom can be embodied by providing the bottom plate of a transparent material. In this connection, the term “transparent” relates to being translucent for light or specific spectra thereof. In particular, the transparency can be configured to allow microscope inspection particularly of the interior of the cell culturing cavity through the bottom.

Preferably, the inlet of the first perfusion channel upwardly extends and upwardly opens, and the inlet of the second perfusion channel upwardly extends and upwardly opens. Thereby, the inlets can upwardly extend by being vertically arranged. Such upwardly extending inlets having a top open end allow for conveniently accessing the perfusion channels. In particular, the perfusion channels can be fed by pipetting or dripping a medium top down into the inlets.

Preferably, the outlet of the first perfusion channel upwardly extends and upwardly opens, and the outlet of the second perfusion channel upwardly extends and upwardly opens. Similarly as the inlets, the outlets can upwardly extend by being vertically arranged. Such upwardly extending outlets having a top open end allow for being conveniently accessed. In particular, a medium can be exited form the outlets, e.g., by being withdrawn.

Preferably, a vasculature network is arranged in the cell culture cavity. Thereby, the cell culture cavity preferably comprises a hydrogel in which the vasculature network is arranged.

Preferably, the cell culture cavity comprises a well portion having an open top end. For example, such well portion can upwardly widening such that it has a comparably large top opening. If the microfluidic cell culture device comprises plates advantageously stacked plates, the well portion can be embodied in the culture plate and/or in the perfusion plate, and/or in a separate further plate.

The well portion may allow for a convenient and efficient access to the cell culturing cavity. For example, by means of such well a hydrogel can be provided into the cell culturing cavity. This can take place using a manual pipette or multi-pipette or even a liquid handling system.

The well portion may also be used to culture cells. In such embodiments, the cell culturing cavity may be a combined well-compartment chamber.

The compartment portion of the cell culture cavity preferably passes over into the well portion of the cell culture cavity. Thereby, preferably the well portion of the cell culture cavity upwardly widens from the compartment portion to the open top end.

Such arrangement combining the compartment and well portions allow for directly accessing the compartment portion providing, e.g., for providing a medium such as a hydrogel into the compartment portion via the well portion. For example, a fluid hydrogel may be dropped via the well portion in the center of the compartment portion, from where it may spread though capillary forces in the compartment portion. Thereby, meniscus of the advancing fluid stops at the interface of the capillary pressure barriers.

Furthermore, such enhanced access configuration allows for adding at least one cell or an aggregate of cells or tissue on top of the hydrogel in the compartment portion. The cell, aggregate of cells or tissue can either be placed directly on the vascular bed or it can be mixed in a hydrogel to mimic the extracellular matrix of the specific tissue. Cell culture medium containing pro-angiogenic compounds, such as VEGF, may then be pipetted either directly on the tissue or on the gel containing the tissue to foster endothelial sprouting and tissue vascularization.

As mentioned, the well portion can also be used for culturing cells. In particular, an organoid preferably is arranged in the well portion of the cell culture cavity. Thereby, the well portion of the cell culture cavity preferably comprises a hydrogel in which the organoid is arranged.

Preferably, the first capillary pressure barrier essentially vertically connects the first perfusion channel with the well portion of the cell culture cavity and the second capillary pressure barrier essentially vertically connects the second perfusion channel with the well portion of the cell culture cavity.

Preferably, at least one of the first perfusion channel and the second perfusion channel has an inclined side wall.

Preferably, the cell culture cavity has a first side compartment portion, and the first capillary pressure barrier essentially vertically connects the first perfusion channel with the first side compartment portion of the cell culture cavity. Such first side compartment portion allows for parallel culturing of cells in the compartment portions.

Preferably, the cell culture cavity has a second side compartment portion, and wherein the second capillary pressure barrier essentially vertically connects the second perfusion channel with the second side compartment portion of the cell culture cavity. Similarly as the first side compartment portion, such second side compartment portion allows for parallel culturing of cells in the compartment portions.

The first and/or second side compartment portions connected to the compartment portion can each contain specific cells, for instance mural cells (pericytes) that will migrate and/or interact with the cells from the compartment portion. This compartment portion can for instance contain mural cells (pericytes or fibroblasts) that mimic the tissue stroma, or contain bacteria or immune cells to an abscess or a lymph node.

The device or its cell culturing cavity may also comprise further side compartments connected to the first or second side compartments, or to one another via further perfusion channel and capillary pressure barriers. Like this a cascade of compartment portions can be generated. Thereby, each compartment portion can mimic another tissue, vascularized or not. Each compartment portion having a vasculature may be filled with a gel and vascular cells, preferably with the specificities of the vasculature of a specific organ, that self-assemble to create a vascular network. Like for the compartment portion, one cell or aggregate of cells, preferably from the organ that correspond to the vasculature of the same organ can be placed on top of the respective vascular bed to be vascularized. It is envisaged that these tissues can be perfused using one or several inlets and one or several outlets.

In an embodiment, two or more side and other compartment portions are located next to each other and horizontally connected with one or more connection channels situated on the same plan than the compartment portions. The compartment portions having a vasculature may be perfused with at least one of the perfusion channel having an inlet on one side of the respective compartment portion and an outlet on the other side of the respective compartment portion.

In another embodiment, vascular cells may be directly mixed with other cells, such as epithelial cells or stromal cells. The cells can self-organize in a configuration that resembles the configuration found in vivo as shown by Cerchiari et al. (PNAS, 2015, 112(7):2287-92). They can be either directly mixed in the compartment portion or in the well portion.

In another further embodiment, the compartment portion with or without the tissue to be vascularized can be created in an array of compartment portions and arranged so that it corresponds to a typical multiwell cell culture format. In particular aspects, the microfluidic cell culture device can be used in a medium or high throughput manner to investigate multicellular interactions, for instance anti-cancer drug screening.

Preferably, the first capillary pressure barrier comprises a first capillary member with at least one hole connecting the first perfusion channel with the cell culture cavity.

Preferably, the second capillary pressure barrier comprises a second capillary member with a hole connecting the second perfusion channel with the cell culture cavity.

The device can have a first sheet establishing the first capillary member of the first capillary pressure barrier and a second sheet establishing the second capillary member of the second capillary pressure barrier. Advantageously, it comprises one sheet establishing both the first capillary member of the first capillary pressure barrier and the second capillary member of the second capillary pressure barrier. The sheet can be embodied by a foil, a plate or the like.

In another aspect, the invention is a use of a microfluidic cell culturing device as described above for the creation of a self assembled vascular network and/or for the creation of vascularized tissues such as spheroids, organoids and tissue biopsies. Such us is a particularly useful and efficient application of the microfluidic cell culturing device.

These and other aspects of the invention will be apparent from and elucidated with reference to the embodiment(s) described hereinafter. Those of ordinary skill in the art will realize that the preceding and following description is illustrative only and is not intended to be in any way limiting.

### BRIEF DESCRIPTION OF THE DRAWINGS

The microfluidic cell culturing device according to the invention and the use according to the invention are described in more detail hereinbelow by way of exemplary embodiments and with reference to the attached drawings, in which:

FIGS. 1A to 1D show a cross sectional view, a top view, a detail Q of the cross sectional view and an isometric view of the cell culture system of a first embodiment of a microfluidic cell culturing device according to the invention;

FIGS. 2A to 2D show a cross sectional view, a top view, a detail R of the cross sectional view and an isometric view of the cell culture system of a second embodiment of a microfluidic cell culturing device according to the invention;

FIGS. 3A to 3D show a cross sectional view, a top view, a detail S of the cross sectional view and an isometric view of the cell culture system of a third embodiment of a microfluidic cell culturing device according to the invention;

FIGS. 4A and 4B show a cross sectional view and an isometric view of the cell culture system of a fourth embodiment of a microfluidic cell culturing device according to the invention;

FIG. 5A to 5C show a cross sectional view, a detail T of the cross sectional view and an isometric view of the cell culture system of the cell culture system of a fifth embodiment of a microfluidic cell culturing device according to the invention being identical to the microfluidic cell culturing device of FIGS. 4A and 4B but comprising a tissue being vascularized and located in the microfluidic cell culturing device on top of a vascularized bed;

FIG. 6A to 6E show a first cross sectional view, a top view, a detail U of the first cross sectional view, an isometric view and a second cross sectional view including tissue being vascularized of the cell culture system of a sixth embodiment of a microfluidic cell culturing device according to the invention;

FIG. 7A to 7E show a cross sectional view, a top view, a detail V of the cross sectional view and an isometric view of the cell culture system of a seventh embodiment of a microfluidic cell culturing device according to the invention, and a top view of a perforated foil located in the sixth embodiment of the microfluidic cell culturing device;

FIG. 8A to 8C show a cross sectional view, a top view and an isometric view of the cell culture system of a eight embodiment of a microfluidic cell culturing device according to the invention;

FIG. 9A to 9D show a first cross sectional view, a top view, a second cross sectional view including several tissues being vascularized, and an isometric view of the cell culture system of an ninth embodiment of a microfluidic cell culturing device according to the invention;

FIG. 10A to 10C show a cross sectional view, an isometric view and a detail W of the cross sectional view of the cell culture system of an tenth embodiment of a microfluidic cell culturing device according to the invention including an epithelial barrier being vascularized;

FIG. 11A to 11C show a cross sectional view, a top view and an isometric view of the cell culture system of a eleventh embodiment of a microfluidic cell culturing device according to the invention;

FIGS. 12A to 12E show a cross sectional view, a top view, a detail X of the cross sectional view, a detail Y of the top view and an isometric view of the cell culture system of a twelfth embodiment of a microfluidic cell culturing device according to the invention;

FIGS. 13A to 13D show a cross sectional view, a top view, a detail Z of the cross sectional view and an isometric view of the cell culture system of an thirteenth embodiment of a cell culturing device according to the invention; and

FIG. 14 shows a representative picture of the formation of a microvascular network in accordance with the present invention.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part.

Like numbers in two or more figures represent the same or similar elements. In particular, in the following description corresponding parts of different embodiments have the same ending. More specifically, reference signs are generally composed of the number of the embodiment of the microfluidic cell culturing device according to the invention followed by two digits representing the respective part or portion thereof.

In the following description of embodiments, generally, the term “cell culture system” refers to an embodiment of a microfluidic cell culturing device, the term “microvasculature compartment” refers to an embodiment of a compartment portion of cell culture cavity, the term “culture well” refers to an embodiment of a well portion of the cell culture cavity, and the term “aperture” refers to an opening establishing an embodiment of a capillary pressure barrier. In the present context, the term “aperture” is also a synonym for interface, e.g. between the gel used in the microvasculature compartment and the perfusion channel, respectively.

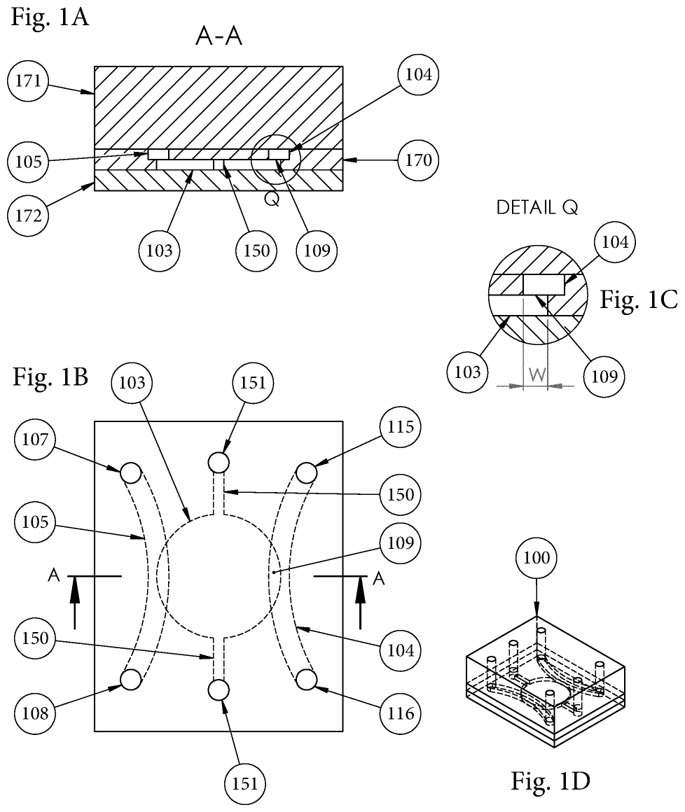

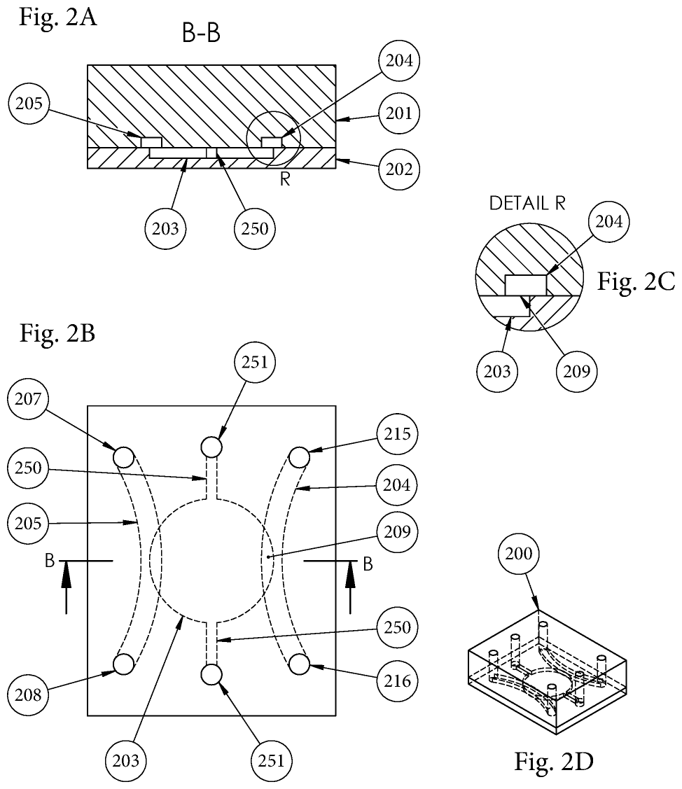

FIGS. 1A to 1D show a cell culture system 100 as a first embodiment of a microfluidic cell culturing device according to the invention. The cell culture system 100 includes cell culture cavity with a microvasculature compartment 103 that is connected to one or more hydrogel channels 150 each with an access hole 151. The microvasculature compartment 103 may be filled with a fluid, such as hydrogel either containing cells or not, that is usually introduced with a pipette via one or more access holes 151 through one or more hydrogel channels 150. The microvasculature compartment 103 is in communication with the external components of the system 100 such as perfusion channels 104, 105, which may contain air, blood, cell culture medium, hydrogel, cells, water, compounds, particulates and/or any other media which are to be delivered to the microvasculature compartment 103 and cellular waste to be extracted from the microvasculature compartment.

In a typical configuration, inlet wells 107, 115 are used as cell culture medium reservoirs and can contain 50 to 300 microliters of liquid, or more. Outlet wells 108, 116 can be of the same size as the inlet wells 107, 115. In such a configuration, a flow induced for instance by a hydrostatic pressure can be created in the perfusion channels 105, 104, for instance by adding a higher level of fluid in the inlet 107,115 than in the outlet wells 108,116 or by placing the cell culture system 100 on a tilting plate as a person skilled in the art would understand. This configuration is of interest for instance when the microvasculature in the compartment 103 is not perfusable, either because the vessels are not yet mature enough or because the cells, such as cells with a disease-specific phenotype, cannot form perfusable vessels or for any other reasons. This configuration can also be used to create an interstitial pressure across the microvasculature compartment 103 that may help induce angiogenic sprouting. The flow in the perfusion channels 104, 105 enables nutrients and oxygen to be delivered to the cells contained in the microvasculature compartment 103 and cellular waste to be extracted from the microvasculature compartment.

In another configuration, used for instance when the vessels in the microvasculature compartment 103 are perfusable, a flow induced for instance by hydrostatic pressure is created between then inlet 107 and the outlet 116. In which case, the outlet 108 and the inlet 115 can be partly or completely blocked for instance by a lid placed on the outlet 108 and the inlet 115 and/or by a barrier placed in the fluid path of the perfusion channel 104 and another one in the fluid path of the perfusion channel 105. The barriers are to be positioned in the fluid path, so that the flow between the inlet 107 and the outlet 116 is not perturbed. It is envisaged that the diameter of the outlet 108 and of the inlet 115 wells is small, typically between 50 to 500 micrometers to act as an air bubble trap.

In an alternative embodiment, the fluid flow can be directed from the perfusion channel 104 to the perfusion channel 105. In this case, the source reservoir 116 of the perfusion channel 104 provides a fluid to one or more microchannels to the microvasculature compartment 103 and the collecting reservoir 107 of the perfusion channel 105 receive the fluid exiting the microvasculature compartment 103. In which case, the outlet 115 and the inlet 108 can be partly or completely blocked for instance by a lid placed on the outlet 108 and the inlet 115 and/or by a barrier placed in the fluid path of the perfusion channel 104 and another one in the fluid path of the perfusion channel 105. The barriers are to be positioned in the fluid path, so that the flow between the inlet 116 and the outlet 107 is not perturbed. It is envisaged that the diameter of the outlet 108 and of the inlet 115 wells is small, typically between 50 to 500 micrometers to act as an air bubble trap.

The microvasculature compartment 103 is located near the bottom part of the cell culture system 100. This is achieved by reducing the height of the bottom plate to a minimum, preferably between 100 and 1000 micrometers. The height of the microvasculature chamber is also limited and has typically a height of 100 to 300 micrometers. This is of importance as the cells and/or tissue to be cultured in the microvasculature compartment 103 will be imaged with a microscope, whose optical path will go through a bottom plate 172, across the microvasculature compartment and a reservoir plate 171. The focal distance between the cells and the objective, usually located below the bottom plate 172, can be maintained as small as possible typically between 0.3 to 2 mm, which enables high resolution imaging.

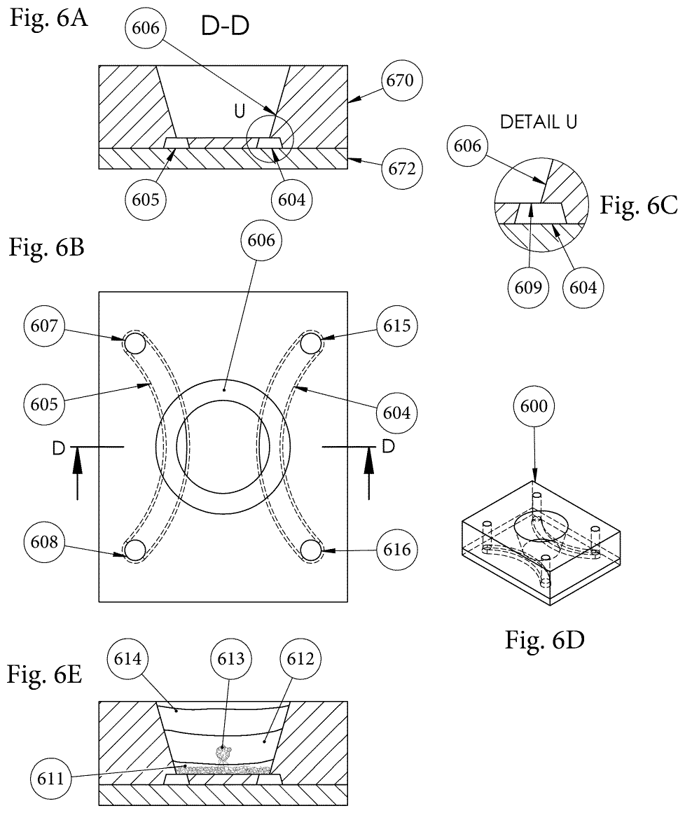

In a sixth embodiment of a cell culturing device according to the invention shown in FIGS. 6, the perfusion channels 604 and 605 are located below the cell culture cavity.

In the following aspects of the capillary pressure barrier are described in more detail.

In general, capillary pressure barriers aim at stopping the progression of the fluid, such as hydrogel, in the microvasculature compartment 103, in particular at the interface between the microvasculature compartment 103 and the perfusion channels 104 and 105 are central to this invention. An aperture 109 of the capillary pressure barrier is the location where the fluid progressing in the microvasculature compartment will be pinned. The aperture is created at the intersection between the perfusion channel 104 and the microvasculature compartment 103 and at the intersection between the perfusion channel 105 and the microvasculature compartment 103. It is envisaged in one embodiment of the cell culture system 100, the aperture is created in a middle plate 170, which presents the important advantage that the aperture size is not defined by the alignment between two plates and is defined during the production of the middle plate 170, for instance by injection molding, a technology that enables high accuracy and thus a high reproducibility of the size of the aperture. It should be noted that the reproducible size of the aperture is important as it directly defines whether the fluid will be pinned or not at the aperture. The size of the aperture 109 also defines the contact area between the hydrogel and the cell culture medium, thus influences the diffusion flux of substances such as nutrients, oxygen and the like in and from the vasculature compartment 103. In other words, the larger the aperture size, the higher is the transport of oxygen, nutrients, growth factors and other chemokines and cytokines. The aperture size is larger than the interface of the capillary barriers of known technologies as the interface is free of trapezoidal posts and does not rely on channel height (phase guide).

In another embodiment of the cell culture system 200, the aperture 209 is created by aligning the bottom plate 202 and the top plate 201 of the cell culture system. In order to avoid a large variation of the size of the aperture 209, alignment pins may be needed to assemble the top plate 201 and the bottom plate 202.

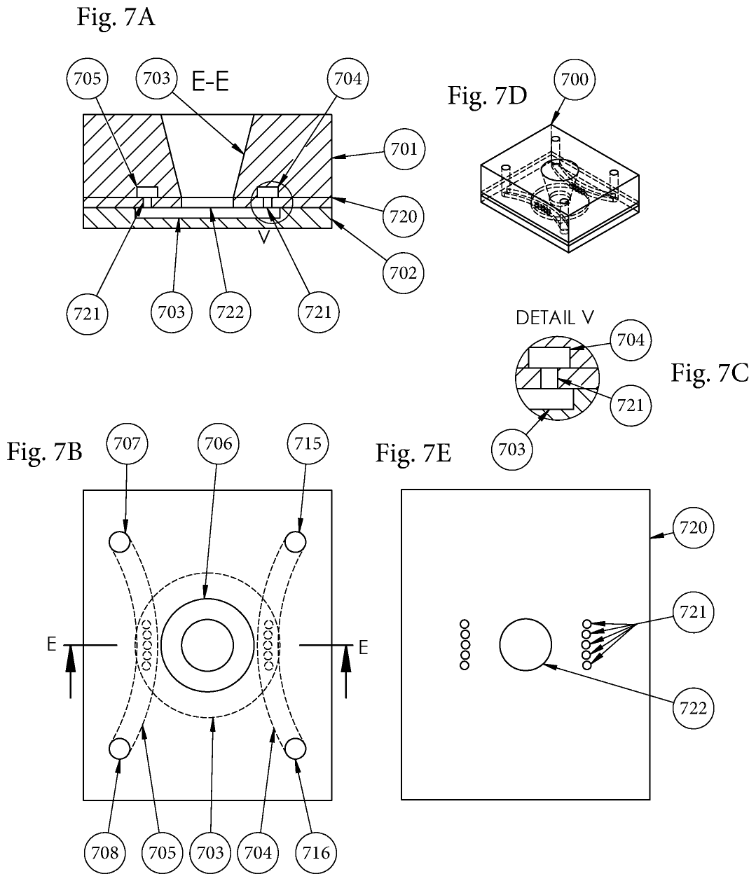

In a sixth embodiment of a cell culturing device according to the invention shown in FIG. 7 having the cell culture system 700, it is envisioned that the aperture of the capillary pressure barrier is created by one or several holes 721 created in a thin foil 720 as a sheet establishing first and second capillary members of the first and second capillary pressure barriers to be placed between the top plate 701 and the bottom plate 702. In a preferred configuration, the holes 721 are of conical shape with the larger diameter near the interface between the microvasculature compartment 703 and the smaller diameter at the interface with the perfusion channels 704 and 705. The fluid meniscus will thus be pinned where the contact angle is the largest at the interface between the hole 721 and the perfusion channels 704 and 705. The hole diameter typically ranges between 100 and 500 micrometers. Other dimensions and shape of the hole can be envisaged.

It is however also conceivable to create several apertures (i.e. more than two) with different sizes. Such an embodiment could be used for instance to mimic an artery (one aperture) that divides into two veins (two apertures). The aperture of the artery would be larger than those of the two veins. Obviously, the size of the aperture is limited by the Young-Laplace law.

In the following aspects in connection with the vascularized tissue are described in more detail.

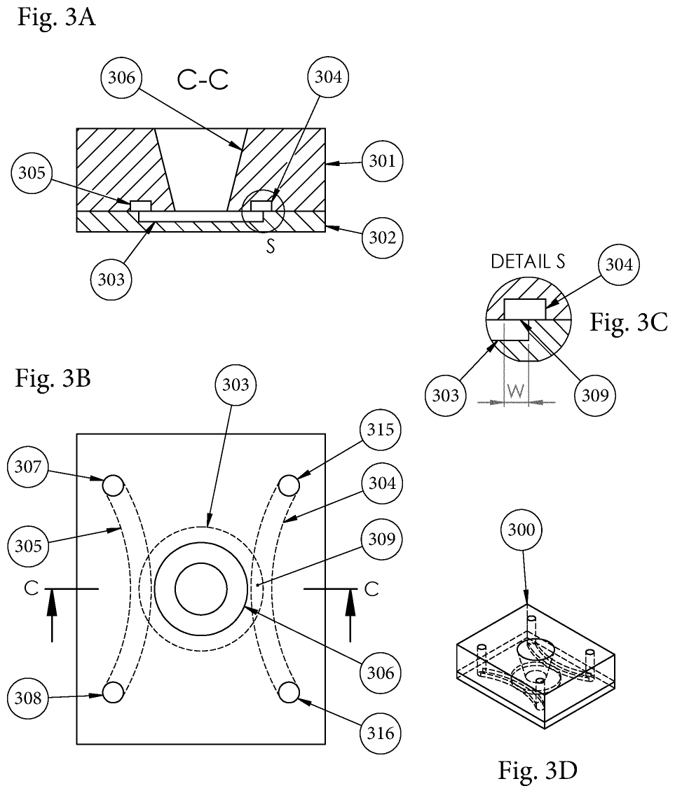

In a third embodiment of a cell culturing device according to the invention shown in FIG. 3 having the cell culture system 300, the reservoir plate 301 contains a through-hole 306 forming a well portion of the cell culture cavity. The through-hole 306 communicates with the microvasculature compartment 303.

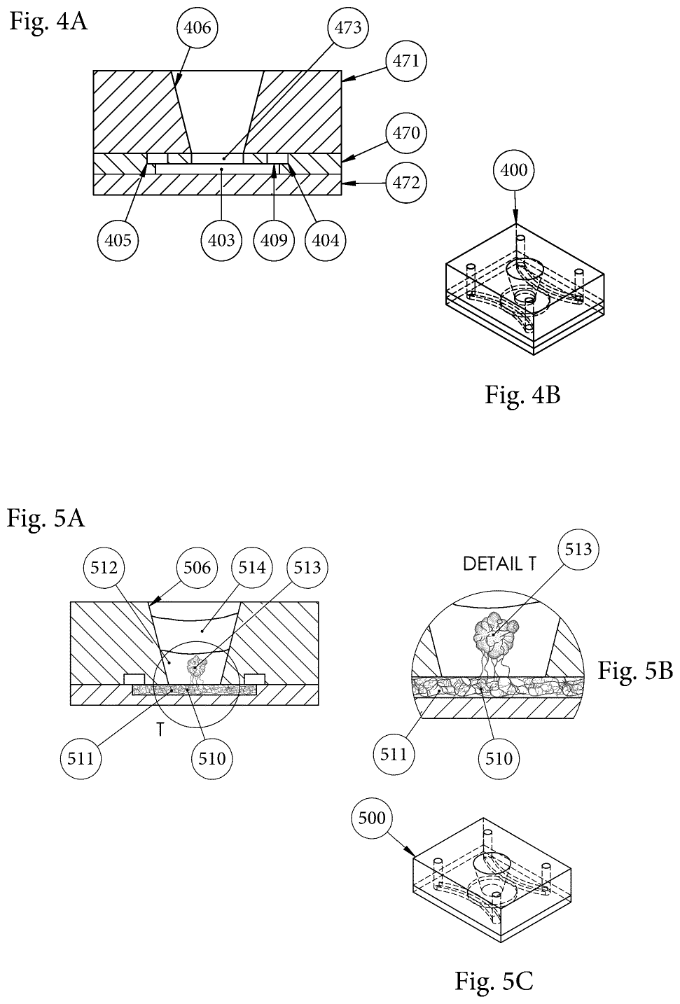

FIG. 4 show a fourth embodiment of a cell culturing device according to the invention having the cell culture system 400. The capillary pressure barriers are created in the middle plate 470 and the latter has an additional hole 473 that communicates between the microvasculature compartment 403 and the culture well 406. In FIG. 5 a fifth embodiment of a cell culturing device according to the invention is shown which is structurally identical to the fourth embodiment but provided with vasculature network 510 and a hydrogel 511 inside the microvasculature compartment, as well as with an organoid 513 and a physiological medium 514 in the through-hole 506.

In another embodiment of the cell culture system 300, 400, 500 the microvasculature compartment 303, 403, 503 can be filled with a fluid, such as a hydrogel 511 from the culture well 306, 406, 506. This can take place using a manual pipette or multipipette or even an automated liquid handling system. The fluid is dropped in the center of the microvasculature compartment, then spreads though capillary forces in the microvasculature compartment. The meniscus of the advancing fluid stops at the interface of the capillary pressure barriers 309, 409.

In the fifth embodiment, the organoid or tissue 513 can be placed in the through-hole or culture well 506 on top of the hydrogel 511 contained in the microvasculature compartment 503. Usually, the tissue 513 is surrounded with a hydrogel 512, itself immerged with physiological medium 514. The tissue 513 can however be cultured on the microvasculature 510 without hydrogel 512 but with the physiological medium 514.

In another embodiment, the microvasculature network 610 can be created directly at the bottom of the culture well 606, by pipetting a hydrogel 611 with cells directly at the bottom of the culture well 606. The perfusion channels 605 and 604 are in this configuration located at the bottom of the culture well, with the apertures 609 of the capillary pressure barriers located at the bottom of the culture well 606. A preferred geometry of the cross-section of the perfusion channels 604 and 605 is an inclined wall to increase the contact angle of the hydrogel meniscus aimed at forcing the hydrogel to be pinned at the interface 609.

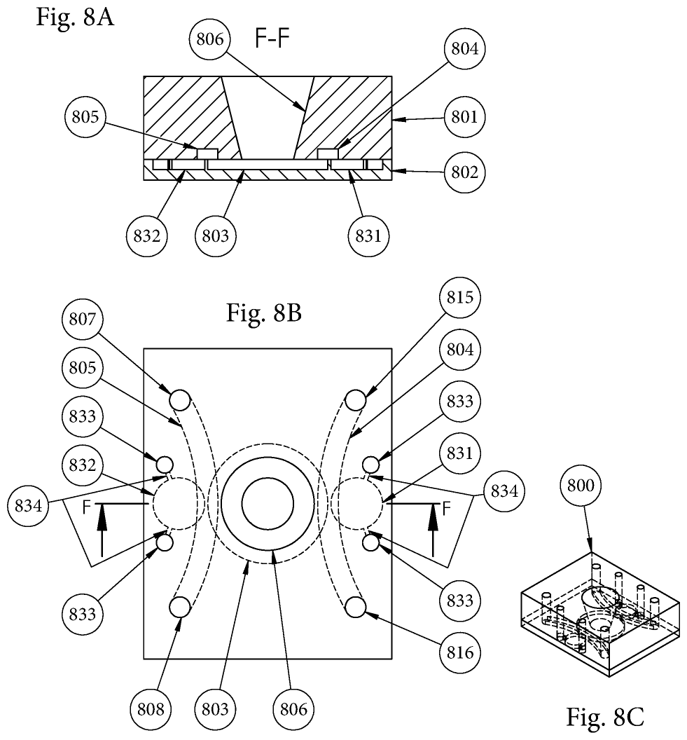

FIG. 8 show a eighth embodiment of a cell culturing device according to the invention having the cell culture system 800. One or more side compartments 831, 832 are in fluidic connection with the microvasculature compartment 803. Each side compartment 831, 832 can be filled individually with hydrogel containing or not cells via access holes 833, which communicate with hydrogel channels 834. A capillary pressure barrier similar to the one located at the interface between the microvasculature compartment 803 and the perfusion channels 804 and 805, stopped the hydrogel meniscus at the interface between the side compartments 831 and 832 and the perfusion channels 804 and 805, respectively. Such configuration can be used to culture specific cells, such as pericytes, in the side compartments as Bichsel et al. teaches us (Bichsel et al., Tissue Engineering Part A 21 (15-16), 2166-2176, 2015).

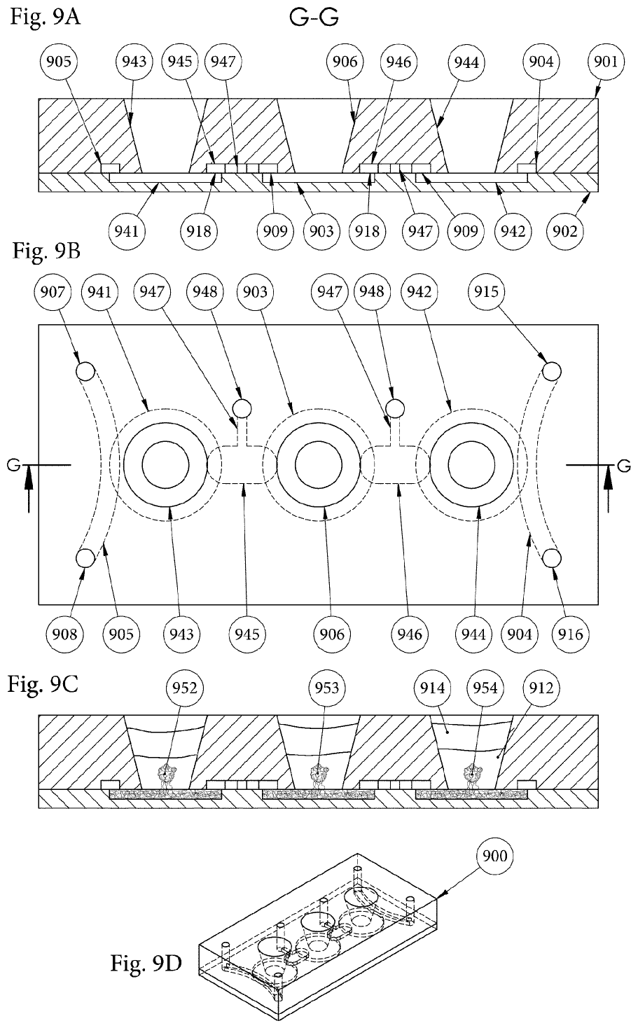

FIG. 9 show a ninth embodiment of a cell culturing device according to the invention having the cell culture system 900. The microvasculature compartment 903 is fluidically connected with one or more microvasculature compartments 941, 942. A connection channel 945, 946 fluidically connects two or more microvasculature compartments 903, 941, 942, and can be filled with hydrogel or physiological medium or the like. One or more vent channels 947 are fluidically connected to the connection channel 945, 946, which serve for instance to allow the air trapped in the connection channel to be removed during its filling via a vent hole 948. In a preferred configuration, the length of the connection channel 945, 946 is short, typically 1 to 2 millimetres. The microvasculature compartment 903 is filled first. In a second step, the microvasculature compartment 941 is filled together with the connection 945 between which no capillary pressure barrier is designed. In such a case, the aperture 918 is designed so that the fluid meniscus is not pinned at the interface between the microvasculature compartment 941 and the connection channel 945. Once all the microvasculature compartments 903, 941, 942 are filled, and the microvasculature networks formed, different tissues, such as from the liver, from the intestine, from the lung can be placed on the culture wells 943, 906, 944 as described earlier.

This configuration enables to create a multi-organs-on-chip with different tissues 952, 953, 954 can be cultured so that the first 952 communicate with the second 953 and both with the third 954. A flow of cell culture medium circulates from the perfusion channel 905 to the perfusion channel 904.

In another embodiment, the type of the vasculature can be organ specific. For instance, the vasculature in 941 can be created by intestinal microvascular endothelial cells, the vasculature in 903 by hepatic endothelial sinusoidal cells, and the vasculature in 942 by lung microvascular endothelial cells. Such a multi-organs-on-chip system would mimic the drug response in vivo. An anticancer drug that is given orally first reaches the gastrointestinal tract for drug absorption into the circulatory system. Further, the drug reaches the liver vasculature, where drug metabolism occurs to convert prodrugs to active metabolites. Finally, these active metabolites reach the target lung tumor microvasculature to exert its anticancer effect.

In another embodiment, the size of the vasculature compartments 941, 903, 942 are designed so that the ratio between tissue vasculatures corresponds to the in-vivo ratio. Vascular heterogeneity between different tissues mostly results from biochemical and mechanical cues in the tissue microenvironment. Therefore, several parameters can be considered to compare two organs, such as the vasculature projected surface, total vascularized area, vessel permeability, volume, endothelial cell turnover and gene expression profile.

In another embodiment, the size of the vasculature compartments 941, 903, 942 are designed so that the ratio between tissue and vasculature corresponds to the in-vivo ratio. Vascular heterogeneity between different tissues mostly results from biochemical and mechanical cues in the tissue microenvironment. Therefore, several parameters can be considered to compare two organs, such as the vasculature projected surface, total vascularized area, vessel permeability, volume, endothelial cell turnover and gene expression profile.

In another embodiment, the size of the vasculature compartments 941, 903, 942 are designed so on one hand that the ratio between tissue and the corresponding vasculature corresponds to the in-vivo ratio and on the other hand that the ratio between the different tissues and their specific vasculature corresponds to the in-vivo ratio. Vascular heterogeneity between different tissues mostly results from biochemical and mechanical cues in the tissue microenvironment. Therefore, several parameters can be considered to compare two organs, such as the vasculature projected surface, total vascularized area, vessel permeability, volume, endothelial cell turnover and gene expression profile.

In another embodiment, the tissues 952, 953, 954 can either be 3D tissues, such as organoids, or a combination between 3D tissues and an epithelial barrier 1060, or only epithelial barriers.

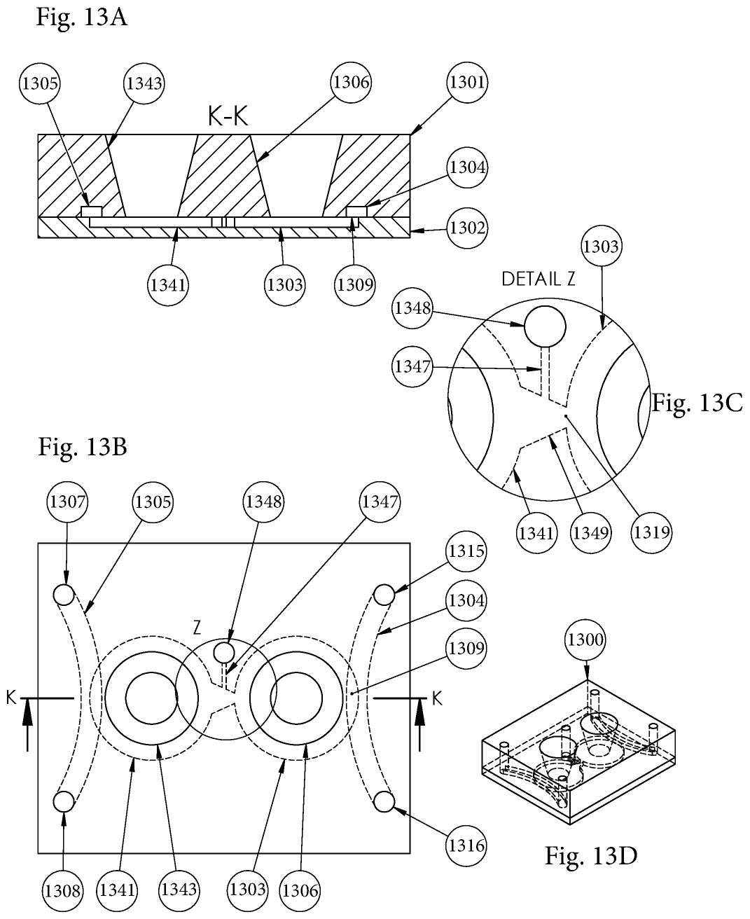

FIG. 13 show a thirteenth embodiment of a cell culturing device according to the invention having the cell culture system 1300. The microvasculature compartment 1303 is fluidically connected to one microvasculature compartment 1341. A connection channel 1349 fluidically connects the microvasculature compartments 1303 and 1341. This is typically done by first filling the compartment 1303 with hydrogel mixed for instance with vascular cells specific to one organ. The fluid meniscus is pinned at the interface between the microvasculature compartment 1303 and the connection channel 1349. This is done by designing one 1319 or several apertures of the connection channel 1349 so that the contact angle increases suddenly at the interface based on the Young Laplace law. Once the first microvasculature compartment is filled the second microvasculature compartment 1341 is filled with hydrogel mixed with vascular cells specific to a second organ. One or more air channel 1347 and air vent 1348 are fluidically connected to the connection channel 1349 to allow the air trapped in the connection channel 1349 to be removed during its filling. This allows the hydrogel of the second microvasculature compartment 1341 to be in contact with the hydrogel of the first microvasculature compartment 1303. As described earlier, the connected microvasculature compartments 1303 and 1341 can be perfused by the perfusion channels. This configuration enables a direct vascularisation between two organ-specific vasculatures, by creating anastomosis between the vasculature and simplifies the microfluidic paths.

In another embodiment, two or more microvasculature compartments can be fluidically connected either using a capillary barrier defined in the XY-plane, such as 909, or the Z-plane, such as 1319. It is also envisaged that both types of capillary barriers can be used simultaneously, typically to increase the surface contact area between the different vasculatures.

In another embodiment, it is also envisaged that perfusion channels can be fluidically connected to the connection channels 945, 946, 1349 for instance to circumvent a vascularised tissue in view to reduce the flow rate in this tissue. This configuration is envisaged to mimic the dual blood supply of the liver, in particular the hepatic artery that directly supply oxygenated blood to the liver from the heart unlike the hepatic portal vein that nutrient-rich blood from the gastrointestinal tract.

In another embodiment, it is envisaged that a hydrostatic pressure is created between the perfusion channel 1305 and the perfusion channel 1304. This can be achieved by a perfusion channel 1305 located at the top of the microvasculature compartment and a second 1304 located at the bottom of the microvasculature compartment. This allows for instance to create a defined interstitial pressure across the microvasculature compartment that will help the creation of perfusable microvessels.

In the following aspects in connection with the vascularized barrier are described in more detail.

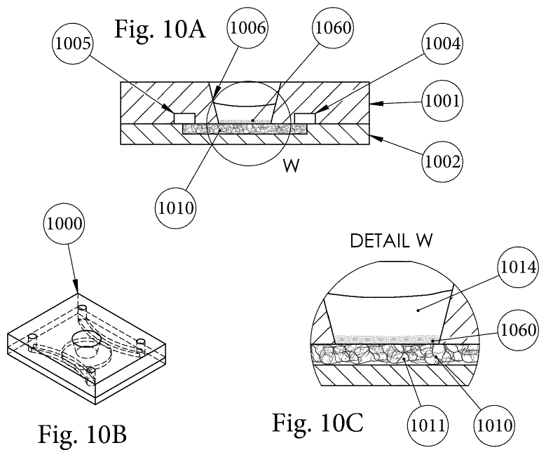

FIG. 10 show a tenth embodiment of a cell culturing device according to the invention having the cell culture system 1000. The tissue 1060 grown on top of the vasculature in view to be vascularized, is an epithelial barrier, such as from the skin, the gut, the lung, etc. The barrier is immerged in a cell culture medium 1014 or culture at the air-liquid interface, such as for the skin or the lung.

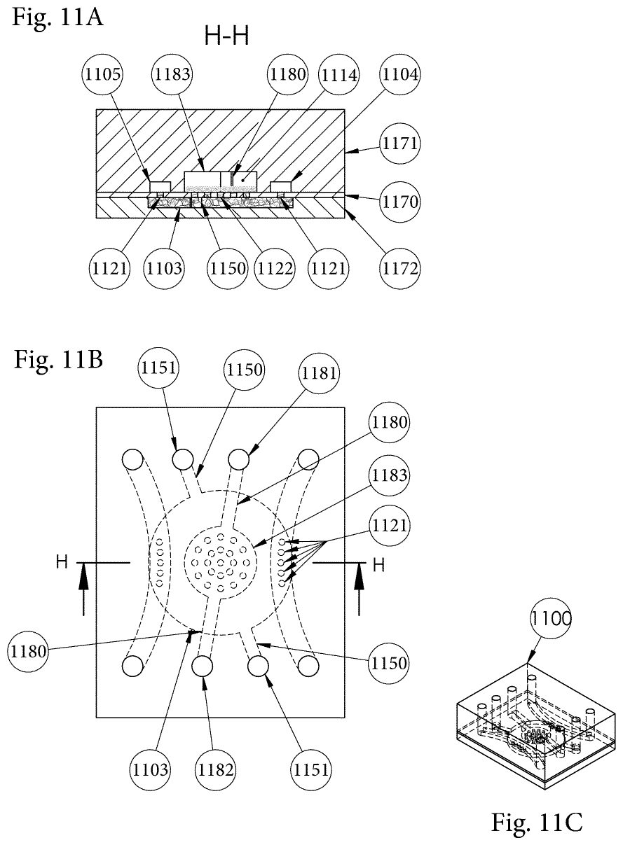

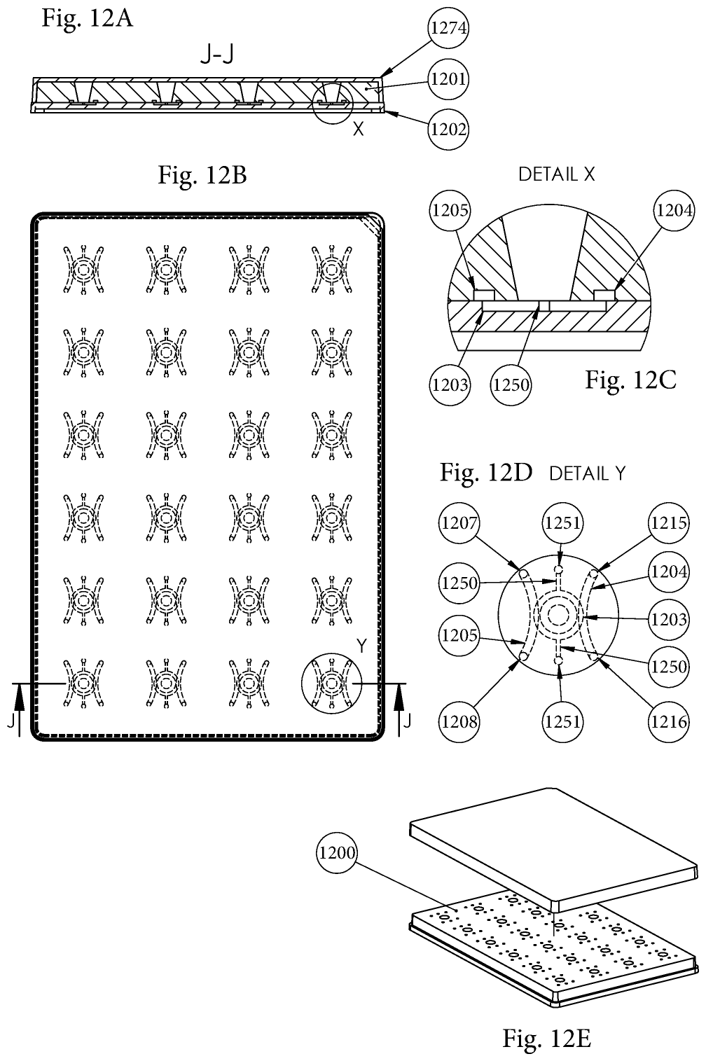

FIG. 11 show a fourth embodiment of a cell culturing device according to the invention having the cell culture system 1100. The epithelial barrier is created in a cell culture chamber 1183 located above the microvasculature compartment 1103, from which it is separated by a thin porous foil 1170. The thin, porous foil 1170 enables the filling of the microvasculature compartment 1103 without hydrogel leaking in the culture chamber 1183 via the thin foil openings 1122. The geometry of these openings 1122 is for instance similar to the thin foil holes 721 so that the fluid meniscus stops at the interface between the openings 1122 and the cell culture chamber 1183. The advantage of this configuration is that a flow can be created on the epithelial barrier for instance to expose it to a continuous supply of nutrients and/or level of oxygen (hypoxia) and to a specific level of shear stress created by the flow of cell culture medium 1114.

In the following aspects in connection with the microfluidic cell culturing device are described in more detail.

The top plate and the bottom plate are preferably made of an essentially non-flexible biocompatible polymer, including but not limited to cyclic olefin copolymer, polystyrene or any other elastomeric or thermoplastic material or other materials like glass, silicon, soft or hard plastic, and the like. However, they can be made of soft material as well, and can be different from each other.

It is envisaged that the cell culture system will be made in a multiwell plate format according to the SLAS guidelines, for instance in a medium-throughput or high-throughput format. The multi-well plate is then composed by 12, 24, 48, 96 or even 384 wells.

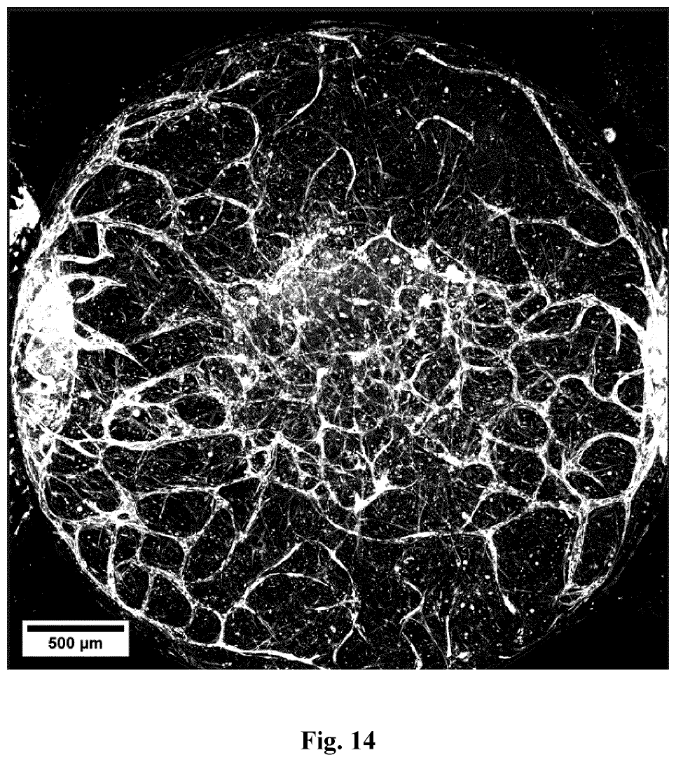

FIG. 14 shows a representative picture of the formation of a microvasculature network between both capillary valves on the left and right side of the central chamber. An increased vessel density in the central region indicates angiogenesis sprouting vessels towards the cancer spheroid, which is located above the vascular bed.

This description and the accompanying drawings that illustrate aspects and embodiments of the present invention should not be taken as limiting-the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

### LIST OF REFERENCE SIGNS

In the following the reference signs of elements depicted in the Figs. are listed. Each of the reference signs contains two parts. A first left part being the number of the embodiment of the cell culturing device according to the invention. In order to reduce the extent of the list, in the following the first left part is represented by xx. A second right part of the reference signs has two digits and represents one specific element of the respective embodiment. Not all of the embodiments of the cell culturing device according to the invention shown in the Figs. do comprise all of the elements listed below. Also, not all elements shown in the Figs. are explicitly recited in the above description of the embodiments above. Rather, the exemplary description of elements in connection with one of the embodiments of the cell culturing device according to the invention does apply to the same element of another embodiment having the corresponding reference sign in the Figs.

| | | | |
|---|---|---|---|
| xx00 | Cell culture system | xx31 | Side compartment right |
| xx01 | Top plate | xx32 | Side compartment left |
| xx02 | Bottom plate | xx33 | Access hole hydrogel side comp. |
| xx03 | Microvasculature compartment | xx34 | Hydrogel channel side compart. |
| xx04 | Perfusion channel right | xx41 | Microvasculature compartment left |
| xx05 | Perfusion channel left | xx42 | Microvasculature compart. right |
| xx06 | Culture well | xx43 | Culture well left |
| xx07 | Inlet well perfusion channel left | xx44 | Culture well right |
| xx08 | Outlet well perfusion channel left | xx45 | Connection channel left |
| xx09 | Aperture | xx46 | Connection channel right |
| xx10 | Vasculature network | xx47 | Air bubble channel |
| xx11 | Hydrogel of the vasculature | xx48 | Air bubble vent |
| xx12 | Hydrogel of the organoid | xx50 | Hydrogel channel |
| xx13 | Organoid | xx51 | Access hole hydrogel channel |
| xx14 | Physiological medium | xx52 | Tissue 1 |
| xx15 | Inlet well perfusion channel right | xx53 | Tissue 2 |
| xx16 | Outlet well perfusion channel right | xx54 | Tissue 3 |
| xx18 | non-pinned aperture | xx60 | Cellular barrier |
| xx20 | Thin foil | xx61 | Perfusion channel |
| xx21 | Thin foil holes | xx70 | Middle plate |
| xx22 | Thin foil opening | xx71 | Reservoir plate |
| xx72 | Bottom part | | |
| xx73 | Well culture hole | | |
| xx74 | Cover | | |
| xx80 | Perfusion channel | | |
| xx81 | Inlet of the perfusion channel | | |
| xx82 | Outlet of the perfusion channel | | |
| xx83 | Cell culture chamber | | |

1. A microfluidic cell culturing device, comprising
a cell culture cavity;
a perfusion channel having an inlet and an outlet; and
a capillary pressure barrier essentially vertically connecting the perfusion channel with the cell culture cavity.

2. The microfluidic cell culturing device of claim 1, wherein the cell culture cavity has a compartment portion.

3. The microfluidic cell culturing device of claim 1, wherein the capillary pressure barrier essentially vertically connects the perfusion channel with the compartment portion of the cell culture cavity.

4. (canceled)

5. The microfluidic cell culturing device of claim 1, wherein the cell culturing cavity comprises a neighboring compartment portion adjacent to the compartment portion.

6. The microfluidic culturing device of claim 1, comprising a capillary pressure compartment barrier essentially vertically connecting the compartment portion and the neighboring compartment portion.

7. The microfluidic cell culturing device of claim 1, having a bottom side essentially horizontally extending.

8. The microfluidic cell culturing device of claim 1, wherein the bottom side is configured such that, in use, the microfluidic cell culturing device seats on the bottom side.

9. The microfluidic cell culturing device of claim 1, comprising a culture plate and a perfusion plate, wherein the culture plate is equipped with the compartment portion of the cell culture cavity and the perfusion plate is equipped with the perfusion channel.

10. The microfluidic cell culturing device of claim 1, wherein the culture plate is mounted on top of the perfusion plate or the perfusion plate is mounted on top of the culture plate.

11. The microfluidic cell culturing device of claim 1, wherein the culture plate abuts the perfusion plate.

12. The microfluidic cell culturing device of claim 1, comprising a bottom plate.

13. The microfluidic cell culturing device of claim 1, wherein the bottom plate abuts the cell culture plate.

14. The microfluidic cell culturing device of claim 1, wherein the capillary pressure barrier has a vertical passage between the perfusion channel and the cell culture cavity.

15. The microfluidic cell culturing device of claim 1, comprising an inflow medium channel which has an inlet and which opens into the cell culture cavity.

16. The microfluidic cell culturing device of claim 1, comprising an outflow medium channel which has an outlet and which opens into the cell culture cavity.

17. The microfluidic cell culturing device of claim 1, wherein the cell culture cavity has a transparent bottom.

18. The microfluidic cell culturing device of claim 1, wherein
the inlet of the perfusion channel upwardly extends and upwardly opens.

19. The microfluidic cell culturing device of claim 1, wherein
the outlet of the perfusion channel upwardly extends and upwardly opens.

20. The microfluidic cell culturing device of claim 1, wherein a vasculature network is arranged in the cell culture cavity.

21. The microfluidic cell culturing device of claim 1, wherein the cell culture cavity comprises a hydrogel in which the vasculature network is arranged.

22. The microfluidic cell culturing device of claim 1, wherein the cell culture cavity comprises a well portion having an open top end.

23. The microfluidic cell culturing device of claim 1, wherein the compartment portion of the cell culture cavity passes over into the well portion of the cell culture cavity.

24. The microfluidic cell culturing device of claim 1, wherein the well portion of the cell culture cavity upwardly widens from the compartment portion to the open top end.

25. The microfluidic cell culture device of claim 1, wherein an organoid is arranged in the well portion of the cell culture cavity.

26. The microfluidic cell culture device of claim 1, wherein the well portion of the cell culture cavity comprises a hydrogel in which the organoid is arranged.

27. The microfluidic cell culture device of claim 1, wherein the capillary pressure barrier essentially vertically connects the perfusion channel with the well portion of the cell culture cavity.

28. The microfluidic cell culture device of claim 1, wherein the perfusion channel.

29. The microfluidic cell culture device of claim 1, wherein the cell culture cavity has a side compartment portion, and wherein the capillary pressure barrier essentially vertically connects the perfusion channel with the side compartment portion of the cell culture cavity.

30. (canceled)

31. The microfluidic cell culture device of claim 1, wherein the capillary pressure barrier comprises a capillary member with at least one hole connecting the perfusion channel with the cell culture cavity.

32. (canceled)

33. Use of a microfluidic cell culturing device according to claim 1 for the creation of a self-assembled vascular network and/or for the creation of vascularized tissues such as spheroids, organoids and tissue biopsies.

34. The microfluidic cell culturing device of claim 1, wherein the perfusion channel at least partially overlaps with the cell culture cavity thereby creating an aperture of the capillary pressure barrier at the intersection between the perfusion channel and the cell culture cavity
